(19) United States
(12) Patent Application Publication
(10) Pub. No.: US 2017/0290890 A1
(43) Pub. Date: Oct. 12, 2017

(54) STIMULATION OF OVARIAN FOLLICLE DEVELOPMENT AND OOCYTE MATURATION

(71) Applicant: The Board of Trustees of the Leland Stanford Junior University, Stanford, CA (US)

(72) Inventors: Yuan Cheng, Palo Alto, CA (US); Aaron J.W. Hsueh, Stanford, CA (US)

(21) Appl. No.: 15/508,854

(22) PCT Filed: Sep. 9, 2015
(86) PCT No.: PCT/US15/49203

§ 371 Date: Mar. 3, 2017

Related U.S. Application Data
(60) Provisional application No. 62/048,748, filed on Sep. 10, 2014.

(51) Int. Cl. A61K 38/24; C12N 5/071; A61K 45/06; A61K 31/53; A61K 35/54

(52) U.S. Cl. CPC A61K 38/24; A61K 31/53; A61K 35/54; A61K 45/06; C12N 5/0682; C12N 2501/727

(57) ABSTRACT

Methods are provided for stimulating ovarian follicles in a mammal through activation of the mTor signaling pathway.

### CROSS REFERENCE

This application is a 371 application and claims the benefit of PCT Application No. PCT/US2015/049203, filed Sep. 9, 2015, which claims benefit of U.S. Provisional Patent Application No. 62/048,748, filed Sep. 10, 2014, which applications are incorporated herein by reference in their entirety.

### GOVERNMENT SUPPORT

This invention was made with Government support under contract HD068158 awarded by the National Institutes of Health. The Government has certain rights in the invention.

### BACKGROUND OF THE INVENTION

The growth and maturation of mammalian germ cells is intricately controlled by hormones; including gonadotropins secreted by the anterior pituitary; and local paracrine factors. The majority of the oocytes within the adult human ovary are maintained in prolonged stage of first meiotic prophase; enveloped by surrounding follicular somatic cells. Periodically, a group of primordial follicles enters a stage of follicular growth. During this time, the oocyte undergoes a large increase in volume, and the number of follicular granulosa cells increases. The maturing oocyte synthesizes paracrine factors that allow the follicle cells to proliferate, and the follicle cells secrete growth and differentiation factors that enhance angiogenesis and allow the oocyte to grow. After progressing to a certain stage, oocytes and their follicles die, unless they are exposed to gonadotropic hormones that prevent somatic cell apoptosis.

Mammalian ovaries consist of follicles as basic functional units. The total number of ovarian follicles is determined early in life, and the depletion of this pool leads to reproductive senescence. Each follicle develops to either ovulate or to undergo degeneration. Individual follicles consist of an innermost oocyte, surrounding granulosa cells, and outer layers of thecal cells. The fate of each follicle is controlled by endocrine as well as paracrine factors. The follicles develop through primordial, primary, and secondary stages before acquiring an antral cavity. At the antral stage a few follicles, under the cyclic gonadotropin stimulation that occurs after puberty, reach the preovulatory stage and become a major source of the cyclic secretion of ovarian estrogens in women of reproductive age. In response to preovulatory gonadotropin surges during each reproductive cycle, the dominant Graafian follicle ovulates to release the mature oocyte for fertilization, whereas the remaining theca and granulosa cells undergo transformation to become the corpus luteum.

Once entering the growing pool, ovarian follicles continue to progress into primary, secondary, and early antral stages with minimal loss. Although FSH treatment is widely used to generate preovulatory follicles in infertile patients mainly by suppressing the apoptosis of early antral follicles, some patients are low responders to FSH treatment because their ovaries contain few early antral follicles as reflected by their elevated serum FSH and lower AMH levels on day 3 of the menstrual cycle.

Throughout the reproductive life, primordial follicles undergo initial recruitment to enter the growing pool of primary follicles. In the human ovary, it is estimated that greater than 120 days are required for the primary follicles to reach the secondary follicle stage, whereas it is estimated that 71 days are needed to grow from the secondary to the early antral stage. Once initiated to enter the growing pool, ovarian follicles progress to reach the antral stage and minimal follicle loss was found until the early antral stage. During cyclic recruitment, increases in circulating FSH allow a cohort of antral follicles to escape apoptotic demise. Among this cohort, a leading follicle emerges as dominant by secreting high levels of estrogens and inhibins to suppress pituitary FSH release. The result is a negative selection of the remaining cohort, leading to its ultimate demise. Concomitantly, increases in local growth factors and vasculature allow a positive selection of the dominant follicle, thus ensuring its final growth and eventual ovulation and luteinization. After cyclic recruitment, it takes only 2 weeks for an antral follicle to become a dominant Graafian follicle. The overall development of human follicles from primordial to preovulatory stages require more than six months.

The development of follicles from the smallest primordial and primary follicles to the largest preovulatory follicles requires different stage-dependent stimulatory and survival factors. Methods of efficiently maturing ovarian follicles from primary through secondary, antral, and preovulatory stages is of great interest, including methods for in vitro and in vivo follicle maturation. The present invention addresses this issue.

### SUMMARY OF THE INVENTION

Compositions and methods are provided for stimulating the growth of mammalian ovarian follicles to a pre-ovulatory stage by contacting the follicles with an effective dose of an agent that activates signaling in the mTor pathway, particularly an agent that directly activates mTor, for a period of time sufficient to grow follicles to a pre-ovulatory state. The contacting may be performed in the absence of physical disruption of the ovary, i.e. the ovary is intact. In some embodiments the ovarian follicles are contacted the agent in an ex vivo culture. In other embodiments the ovarian follicles are contacted with the agent in vivo. Where the contacting is performed in vivo, the agent may be administered locally to the ovary, e.g. to women suffering from premature ovarian failure, women suffering from polycystic ovarian syndrome, middle-aged infertile women, etc. The effective dose is a dose that allows ovarian follicles to undergo sufficient growth to reach the pre-ovulatory stage.

A method of promoting the development of mature oocytes is provided, comprising contacting ovarian tissue, including without limitation an intact ovary, in vivo or in vitro with an effective dose of an agent that activates signaling in the mechanistic target of rapamycin (mTor) pathway for a period of time sufficient to promote the development of a mature oocyte. The mature oocyte may be contained within a pre-ovulatory follicle. The pre-ovulatory follicle may be a secondary follicle or an antral follicle. The ovarian tissue may be human, or may be a mammal selected from the group consisting of mice, canines, felines, rabbits, pigs, cows, buffalos, sheep, horses, pandas, chimpanzees and gorillas.

A method of increasing phosphorylation of ribosomal S6 kinase 1 (S6K1) and ribosomal protein S6 (rpS6) in ovarian tissue, including without limitation an intact ovary, is provided, comprising contacting ovarian tissue in vivo or in vitro with an effective dose of an agent that activates signaling in the mechanistic target of rapamycin (mTor) pathway for a period of time sufficient to increase phosphorylation of ribosomal S6 kinase 1 (S6K1) and ribosomal protein S6. The ovarian tissue may be human, or may be a mammal selected from the group consisting of mice, canines, felines, rabbits, pigs, cows, buffalos, sheep, horses, pandas, chimpanzees and gorillas.

Agents of interest for the methods of promoting growth of ovarian follicles to a pre-ovulatory state, promoting development of mature oocytes, and/or increasing phosphorylation of ribosomal S6 kinase 1 (S6K1) and ribosomal protein S6 (rpS6) in ovarian tissue, include an agent directly activates mTor, including without limitation one or more of MHY1485, 3BDO, and CL316,243. The dosage may be from 0.1 μM to about 1 mM; optionally for a period of from one hour to four days.

Methods of the invention may further comprise, following the contacting step, performing a step of contacting the follicle with FSH or an analog thereof in a dose and for a time effective to induce oocyte maturation. The methods may further comprise following the contacting step, performing a step of harvesting the follicle and optionally transplantation of the activated follicles to an in in vivo recipient. The recipient may be autologous to the ovarian follicle. Optionally an LH agonist is administered to the recipient following implantation.

Methods of the invention may further comprise contacting the follicle with an effective dose of at least one of PTEN inhibitor and a PI3 kinase activator with the agent that directly activates mTor.

In some embodiments, the invention provides for use in the preparation of a medicament of an agent that activates signaling in the mTor pathway for the treatment of mammalian female infertility, including without limitation human females. The female infertility may be due to a condition selected from the group consisting of premature ovarian failure, perimenopause, FSH low responsiveness, polycystic ovarian syndrome, diminished ovarian reserve and age-related infertility. The agent may directly activate mTor, including without limitation one or more of MHY1485, 3BDO, and CL316,243.

Mechanistic target of rapamycin (mTOR) is an atypical serine/threonine kinase and mTOR signaling is important in regulating cell growth and proliferation. Agents of interest for activation of mTor include, without limitation, small molecules such as MHY1485, 3-benzyl-5-((2-nitrophenoxy) methyl)-dihydrofuran-2(3H)-one (3BDO), CL316,243, etc.

The methods of the invention may be further combined with the step of contacting the ovarian follicles with additional agents that activate growth of ovarian follicles, including without limitation contacting the follicles with at least one of a phosphatase and tensin homolog (PTEN) inhibitor, and a phosphatidylinositol 3-kinase (PI3 kinase) activator, which provides for an additive or synergistic effect.

In some embodiments of the invention, the exposure is performed in vitro, e.g. in an organ or tissue culture, where at least one ovarian follicle is exposed to an effective dose of an agent that activates signaling in the mTor pathway. The treated follicle may be utilized for in vitro purposes, for example for in vitro fertilization, generation of embryonic stem cells, etc., or may be transplanted to provide for in vivo uses. Transplantation modes of interest include, without limitation, transplantation of one or more follicles, including follicles present in an ovary that has not been physically disrupted, to a kidney capsule, to a subcutaneous site, near the fallopian tubes, to an ovarian site, e.g. where one ovary has been retained and one has been removed for ex vivo treatment, the one or more treated follicles may be transplanted to the site of the remaining ovary.

In some embodiments, in vitro treatment is followed by ovarian transplantation to activate follicles for the generation of preovulatory oocytes, which may be followed by in vitro or in vivo fertilization.

Individuals of interest include endangered species, economically important animals, women suffering from premature ovarian failure, women suffering from polycystic ovarian syndrome, middle-aged infertile women, follicles derived from human embryonic stem cells and primordial germ cells, and the like. In other embodiments, the exposure is performed in vivo, locally, e.g. by intra-ovarian injection, or systemically administered to an individual.

Following exposure of an individual to an effective dose of an agent that activates signaling in the mTor pathway, the individual may be treated with follicular stimulating hormone (FSH) or FSH analogs, including recombinant FSH, naturally occurring FSH in an in vivo host animal, FSH analogs, e.g. FSH-CTP, pegylated FSH, and the like, at a concentration that is effective to initiate follicular growth.

Where the follicles have been stimulated to the pre-ovulatory stage stage, the individual may be treated with luteinizing hormone (LH) or an agonist thereof, which agonists specifically include chorionic gonadotropins, e.g. equine chorionic gonadotropin (eCG), human chorionic gonadotropin (HCG), etc., at an ovulatory dose. In addition, the follicles may be exposed in vivo or in vitro to one or more of c-kit ligand, neurotrophins, vascular endothelial growth factor (VEGF), bone morphogenetic protein (BMP)-4, BMP7, leukemia inhibitory factor, basic FGF, keratinocyte growth factor; and the like.

The period of time effective for stimulation with an effective dose of an agent that activates signaling in the mTor pathway according to the methods of the invention is usually at least about one hour and not more than about 5 days, and may be at least about 12 hours and not more than about 4 days, e.g. 2, 3, or 4 days.

### BRIEF DESCRIPTION OF THE FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1D. Treatment of ovaries with MHY1485 increased phosphorylation of mTOR pathway proteins and promoted secondary follicle development in vitro. (FIG. 1A) Treatment of ovaries with MHY1485 increased phosphorylation of mTOR as well as S6K1 and rpS6. Ovaries from day 10 mice were treated with MHY1485 for 3 h before immunoblotting. (FIG. 1B) Ovarian weight changes. Paired ovaries from day 10 mice were incubated with MHY1485 with media changes at day 2 of culture. At the end of 4 days of incubation, ovaries were fixed before weighing, followed by histological analyses. Numbers in parentheses denote number of ovaries used. (FIG. 1C) Ovarian histology; bars: 100 μm. (FIG. 1D) Follicle dynamics.

FIG. 2A-2C. Short-term treatment of ovaries with MHY1485 followed by allo-transplantation promoted secondary follicle growth to the antral stage in ovarian grafts. (FIG. 2A) Graft weight changes. Ovaries from day 10 mice were incubated with MHY1485 for 2 days, before grafting into adult ovariectomized hosts treated daily with FSH for 5 days. At the end of grafting, graft weights were determined and histological analyses were performed. Numbers in parentheses indicate number of grafts used. (FIG. 2B) Ovarian histology; bars: 100 um. (FIG. 2C) Follicle dynamics. PO: preovulatory.

FIG. 3A-2C. Treatment with MHY1485 and subsequent grafting allowed the derivation of mature oocytes and healthy offspring. (FIG. 3A) Early embryonic development of oocytes after mTOR activator treatment. Ovaries were treated with MHY1485 for 2 days to activate follicles, followed by grafting into hosts for 5 days. Hosts were then treated with eCG and hCG. At 12 h after hCG injection, mature oocytes were obtained and fertilized with sperm before culturing for 4 days. (FIG. 3B) Percentage of oocytes developed into each embryonic stage. Early embryonic development for mice at 25 days of age served as controls. (FIG. 3C) Some 2-cell stage embryos were transferred into pseudopregnant hosts and pups were delivered.

FIG. 4A-2C. Additive effects of mTOR activation and AKT stimulation on follicle growth. (FIG. 4A) Graft weight increases. Ovaries from day 10 mice were incubated with IVA drugs with or without MHY1485. Ovaries were then grafted into hosts treated daily with FSH for 5 days before determination of graft weights. (FIG. 4B) Ovarian histology; bars: 100 μm. (FIG. 4C) Follicle dynamics. PO: preovulatory.

### DESCRIPTION OF THE SPECIFIC EMBODIMENTS

Compositions and methods are provided for modulating the growth and maturation of mammalian ovarian follicles. By exposing follicles to an effective dose of at least one of an agent that activates mTor, follicle growth and consequent oocyte maturation can be manipulated.

The methods of the invention find use in a wide variety of animal species, particularly including mammalian species. Animal models, particularly small mammals, e.g. murine, lagomorpha, etc. are of interest for experimental investigations. Other animal species may benefit from improvements in in vitro fertilization, e.g. horses, cattle, rare zoo animals such as panda bears, large cats, etc. Humans are of particular interest for enhancing oocyte maturation, including methods of in vitro fertilization. Individuals of interest for treatment with the methods of the invention include, without limitation, those suffering from premature ovarian failure, peri-menopause, FSH low responsiveness, polycystic ovarian syndrome, age-related infertility, i.e. woman greater than 40 years of age, etc.

Embodiments of the invention can include ovarian follicles of numerous species of mammals. The invention should be understood not to be limited to the species of mammals cited by the specific examples within this patent application. Embodiments of the invention, for example, may include fresh or frozen-thawed follicles of animals having commercial value for meat or dairy production such as swine, bovids, ovids, equids, buffalo, or the like (naturally the mammals used for meat or dairy production may vary from culture to culture). It may also include ovarian follicles from individuals having rare or uncommon attribute(s), such as morphological characteristics including weight, size, or conformation, or other desired characteristics such as speed, agility, intellect, or the like. It may include ovarian follicles from deceased donors, or from rare or exotic mammals, such as zoological specimens or endangered species. Embodiments of the invention may also include fresh or frozen-thawed ovarian follicles collected from primates, including but not limited to, chimpanzees, gorillas, or the like, and may also ovarian follicles from marine mammals, such as whales or porpoises.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Ovarian Follicle.

An ovarian follicle is the basic unit of female reproductive biology and is composed of roughly spherical aggregations of cells found in the ovary. A follicle contains a single oocyte. Follicles are periodically initiated to grow and develop, culminating in ovulation of usually a single competent oocyte. The cells of the ovarian follicle are the oocyte, granulosa cells and the cells of the internal and external theca layers. The oocyte in a follicle is in the stage of a primary oocyte. The nucleus of such an oocyte is called a germinal vesicle. Granulosa cells within the follicle surround the oocyte; their numbers increase in response to gonadotropins. They also produce peptides involved in ovarian hormone synthesis regulation. Follicle-stimulating hormone (FSH) acts on granulosa cells to express luteinizing hormone (LH) receptors on the cell surface. The granulosa cells, in turn, are enclosed in a thin layer of extracellular matrix—the follicular basement membrane or basal lamina. Outside the basal lamina, the layers theca interna and theca externa are found.

Ovarian In Vitro Culture.

Methods are known in the art for culturing mammalian ovaries or fragments thereof, which fragments for the purposes of the present invention will include at least one follicle. Typically all or a portion of an ovary is placed in tissue culture medium, which medium may include factors useful in the growth or maintenance of the follicle cells, and which, as described herein, further comprise an effective dose of at least an agent that activates signaling in the mTor pathway. See the Examples provided herein. Additional description may be found, inter alia, (each of which reference is herein specifically incorporated by reference) at Hoyer et al. (2007) Birth Defects Res B Dev Reprod Toxicol. 80(2):113-25. In vitro culture of canine ovaries is described by Luvoni et al. (2005) Theriogenology.; 63(1):41-59. Culture of bovine follicles is described by Hansel (2003) Anim Reprod Sci.; 79(3-4):191-201.

A review of in vitro ovarian tissue and organ culture may be found in Devine et al. (2002) Front Biosci. 7:d1979-89; and in Smitz et al. (2002) Reproduction. 123(2):185-202. Whole ovaries from fetal or neonatal rodents have been incubated in organ culture systems. Adaptations of this technique include incubation of ovaries in a chamber continuously perfused with medium or perfusion of medium through the intact vasculature. Another approach has been to culture individual follicles isolated by enzymatic or mechanical dissociation. Cryopreservation of human primordial and primary ovarian follicles is described by Hovatta (2000) Mol Cell Endocrinol. 169(1-2):95-7.

Ovarian Transplantation.

Ovarian transplantation to the kidney is a well-established procedure in animal studies. Autologous transplantation of ovarian cortical tissue has been widely reported in humans, particularly in the context of women undergoing sterilizing cancer therapy or surgery. Ovarian tissue may be transplanted fresh, or after cryo-preservation. For a review, see Grynberg et al. (2012) Fertil. Steril. 97(6):1260-8, herein specifically incorporated by reference.

Mtor.

The mechanistic target of rapamycin (mTOR) is an atypical serine/threonine kinase. The genetic sequences may be accessed at Genbank, where the human sequence is represented by NM_004958.3, and for protein, NP_004949.1. mTor can is present in two distinct complexes. mTOR complex 1 (mTORC1) is composed of mTOR, Raptor, GβL (mLST8), and Deptor and is partially inhibited by rapamycin. mTORC1 integrates multiple signals reflecting the availability of growth factors, nutrients, or energy to promote either cellular growth when conditions are favorable or catabolic processes during stress or when conditions are unfavorable. Growth factors and hormones (e.g. insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling. Amino acid availability is signaled to mTORC1 via a pathway involving the Rag and Regulator (LAMTOR1-3) proteins. Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. The mTOR complex 2 (mTORC2) is composed of mTOR, Rictor, GβL, Sin1, PRR5/Protor-1, and Deptor and promotes cellular survival by activating Akt. mTORC2 also regulates cytoskeletal dynamics by activating PKCα and regulates ion transport and growth via SGK1 phosphorylation. Aberrant mTOR signaling is involved in many disease states including cancer, cardiovascular disease, and metabolic disorders.

Agents that Activate MTor Signaling.

In addition to growth factors and hormones, such as insulin, a number of small molecule mTor activators are known and used in the art, including, without limitation, 3-benzyl-5-((2-nitrophenoxy) methyl)-dihydrofuran-2(3H)-one (3BDO) (see Ge et al. (2014) Autophagy 10(6):957-71); 4,6-Di-4-morpholinyl-N-(4-nitrophenyl)-1,3,5-triazin-2-amine (MHY1485) (see Choi et al. (2012) PLoS ONE 7:8 special section p1); 5-[(2R)-2-[[(2R)-2-(3-Chlorophenyl)-2-hydroxyethyl]amino]propyl]-1,3-benzodioxole-2,2-dicarboxylic acid (CL316,243) (see Miniaci et al. (2013) Pflugers Arch. 2013 April; 465(4):509-16).

The effective concentration of MHY1485 for in vitro culture may be from about 0.1 μM, about 1 μM, about 10 μM, about 50 μM, and not more than about 1 mM. For in vivo purposes the dose may vary depending on the individual and the manner of dosing, e.g. it may be desirable to localize the agent so as to achieve a higher concentration in the targeted tissue. Effective concentrations for other agents may be based on a determination of relative strength compared to MHY1485, or determined empirically.

FSH.

Follicle-stimulating hormone (FSH) is a hormone synthesized and secreted by gonadotropes in the anterior pituitary gland. FSH regulates the development, growth, pubertal maturation, and reproductive processes of the human body. FSH and Luteinizing hormone (LH) act synergistically in reproduction. In females, in the ovary FSH stimulates the growth of immature follicles to maturation. As the follicle grows, it releases inhibin, which shuts off the FSH production.

FSH is a dimeric glycoprotein. The alpha subunits of LH, FSH, TSH, and hCG are identical, and contain 92 amino acids. FSH has a beta subunit of 118 amino acids (FSHB), which confers its specific biologic action and is responsible for interaction with the FSH-receptor. The half-life of native FSH is 3-4 hours. Its molecular wt is 30000.

Various formulations of FSH are available for clinical use. It is used commonly in infertility therapy to stimulate follicular development, notably in IVF therapy, as well as with interuterine insemination (IUI). FSH is available mixed with LH in the form of Pergonal or Menopur, and other more purified forms of urinary gonadotropins, as well as in a pure forms as recombinant FSH (Gonal F, Follistim), and as Follistim AQ, Gonal-F, Gonal-f RFF, Gonal-f RFF Pen.

Analogs of FSH are also clinically useful, which analogs include all biologically active mutant forms, e.g. where one, two, three or more amino acids are altered from the native form, PEGylated FSH, single chain bi-functional mutants, FSH-CTP, and the like. In an effort to enhance ovarian response several long-acting FSH therapies have been developed including an FSH-CTP (Corifollitropin alfa), where the FSH beta subunits are linked by the C-terminal peptide (CTP) moiety from human chorionic gonadotropin (hCG); and single-chain bi-functional VEGF-FSH-CTP (VFC) analog. FSH-CTP has a longer half-life in vivo, and may be administered, for example, with an interval of from one to four weeks between doses. See, for example, Lapolt et al. (1992) Endocrinology 131:2514-2520; and Devroey et al. (2004) The Journal of Clinical Endocrinology & Metabolism Vol. 89, No. 5 2062-2070, each herein specifically incorporated by reference.

LH and Agonists.

LH is a heterodimeric glycoprotein. Its structure is similar to that of the other glycoprotein hormones, follicle-stimulating hormone (FSH), thyroid-stimulating hormone (TSH), and human chorionic gonadotropin (hCG). The protein dimer contains 2 glycopeptidic subunits, labeled alpha and beta subunits, that are non-covalently associated. The alpha subunits of LH, FSH, TSH, and hCG are identical, and contain 92 amino acids in human but 96 amino acids in almost all other vertebrate species. The beta subunits vary. LH has a beta subunit of 120 amino acids (LHB) that confers its specific biologic action and is responsible for the specificity of the interaction with the LH receptor. This beta subunit if highly homologous to the beta subunit of hCG and both stimulate the same receptor. LH is available mixed with FSH in the form of Pergonal, and other forms of urinary gonadotropins Recombinant LH is available as lutropin alfa (Luveris). All these medications are administered parenterally.

Often, hCG medication is used as an LH substitute because it activates the same receptor, is less costly, and has a longer half-life than LH. Human chorionic gonadotropin is a glycoprotein of 244 amino acids. The β-subunit of hCG gonadotropin contains 145 amino acids. Like other gonadotropins, hCG can be extracted from urine or by genetic modification. Pregnyl, Follutein, Profasi, Choragon and Novarel use the former method, derived from the urine of pregnant women. Ovidrel is a product of recombinant DNA. As an alternative, equine chorionic gonadotropin (eCG) is a gonadotropic hormone produced in the chorion of pregnant mares.

PTEN Inhibitor.

The polypeptide PTEN (phosphatase with TENsin homology) was identified as a tumor suppressor that is mutated in a large number of cancers at high frequency. The protein encoded this gene is a phosphatidylinositol-3,4,5-trisphosphate 3-phosphatase. It contains a tensin like domain as well as a catalytic domain similar to that of the dual specificity protein tyrosine phosphatases. Unlike most of the protein tyrosine phosphatases, this protein preferentially dephosphorylates phosphoinositide substrates. It negatively regulates intracellular levels of phosphatidylinositol-3,4,5-trisphosphate in cells and functions as a tumor suppressor by negatively regulating AKT/PKB signaling pathway. The genetic sequence of the human protein may be found in Genbank, accession number NM_000314, as described by Volinia et al. (2008) PLoS ONE 3 (10), E3380; Li et al. (1997) Cancer Res. 57 (11), 2124-2129; Steck et al. (1997) Nat. Genet. 15 (4), 356-362; and Li et al. (1997) Science 275 (5308), 1943-1947, each herein specifically incorporated by reference. PTEN inhibitors of interest may have an IC₅₀ of from about 0.1 nM to about 100 μM, and may be from about 1 nm to about 10 μM, of from about 10 nM to about 1 μM, of from about 1 nM to about 100 nM.

A number of known PTEN inhibitors are known in the art, including without limitation, bisperoxovanadium compounds (see, for example, Schmid et al. (2004) FEBS Lett. 566(1-3):35-8). Included are potassium bisperoxo(bipyridine)oxovanadate (V), which inhibits PTEN at an IC₅₀=18 nM; dipotassium bisperoxo(5-hydroxypyridine-2-carboxyl)oxovanadate (V), which inhibits PTEN at an IC₅₀=14 nM; potassium bisperoxo (1,10-phenanthroline)oxovanadate (V) which inhibits PTEN at an IC₅₀=38 nM; dipotassium bisperoxo(picolinato)oxovanadate (V) which inhibits PTEN at an IC₅₀=31 nM; N-(2-Hydroxy-3-methoxy-5-dimethylamino)benzyl, N′-(2-(4-nitrophenethyl)), N″-methylamine which inhibits the CDC25 phosphatase family; dephostatin which is a competitive PTP inhibitor; monoperoxo(picolinato)oxovanadate(V) which is a PTP inhibitor (IC₅₀=18 μM); and sodium orthovanadate, which is a broad-spectrum inhibitor of phosphatases.

Additional PTEN inhibitors are described by, inter alia, Myers et al. (1998) PNAS 95:13513-13518; by Garlich et al., WO/2005/097119; and by Rosivatz et al. (2007) ACS Chem. Biol., 1, 780-790.

Alternatively, inhibitors of PTEN may be identified by compound screening for agents, e.g. polynucleotides, antibodies, small molecules, etc., that inhibit the enzymatic activity of PTEN, which is known to have phosphatase activity. Compound screening may be performed using an in vitro model, a genetically altered cell or animal or purified PTEN1 protein. One can identify ligands or substrates that bind to or inhibit the phosphatase activity. A wide variety of assays may be used for this purpose, including labeled in vitro protein-protein binding assays, electrophoretic mobility shift assays, immunoassays for protein binding, and the like. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

PI3K activator. Phosphoinositide 3-kinases (PI 3-kinases or PI3Ks) are a family of enzymes involved in cellular functions such as cell growth, proliferation, differentiation, motility, survival and intracellular trafficking, which are capable of phosphorylating the 3 position hydroxyl group of the inositol ring of phosphatidylinositol (PtdIns).

Class I PI3Ks are responsible for the production of Phosphatidylinositol 3-phosphate (PI(3)P), Phosphatidylinositol (3,4)-bisphosphate (PI(3,4)P₂) and Phosphatidylinositol (3,4,5)-trisphosphate (PI(3,4,5)P₃. The PI3K is activated by G-protein coupled receptors and tyrosine kinase receptors.

Class I PI3K are heterodimeric molecules composed of a regulatory and a catalytic subunit; which are further divided between IA and IB subsets on sequence similarity. Class I PI 3-kinases are composed of a catalytic subunit known as p110 and a regulatory subunit either related to p85 or p101. The p85 subunits contain SH2 and SH3 domains.

Activators of PI3K increase the activity of the enzyme. Activators of interest include, without limitation the cell-permeable phospho-peptide (740Y-P), which is capable of binding to the SH2 domain of the p85 regulatory subunit of PI3K to stimulate enzyme activity (commercially available peptide, RQIKIWFQNRRMKWKKSDGGYMDMS, Modifications: Tyr-25=pTyr). Other activators include fMLP (see Inoue T, Meyer T (2008) Synthetic Activation of Endogenous PI3K and Rac Identifies an AND-Gate Switch for Cell Polarization and Migration. PLoS ONE 3(8): e3068. Also see Bastian et al., Mol Cancer Res 2006; 4(6). June 2006; Park et al. Toxicology Toxicology Volume 265, Issue 3, 30 Nov. 2009, Pages 80-86, herein incorporated by reference)

Candidates for Therapy.

Any female human subject who possesses viable ovarian follicles is a candidate for therapy with the methods of the invention. Typically, the subject will suffer from some form of infertility, including premature ovarian failure. For instance, the subject may experience normal oocyte production but have an impediment to fertilization, as in, e.g. PCOS or PCOS-like ovaries. The methods of the invention may be especially useful in women who are not suitable candidates for traditional in vitro fertilization techniques involving an ovarian stimulation protocol. Included are patients with low responses to the conventional FSH treatment.

As described above, the methods of the invention are also useful in the treatment of infertility with various non-human animals, usually mammals, e.g. equines, canines, bovines, etc.

Premature ovarian failure (POF) occurs in 1% of women. The known causes for POF include genetic aberrations involving the X chromosome or autosomes as well as autoimmune ovarian damages. Presently, the only proven means for infertility treatment in POF patients involve assisted conception with donated oocytes. Although embryo cryopreservation, ovarian cryopreservation, and oocyte cryopreservation hold promise in cases where ovarian failure is foreseeable as in women undergoing cancer treatments, there are few other options. Due to heterogeneity of POF etiology, varying amounts of residual primordial follicle are still present in patients' ovaries for activation.

The degrees of ovarian follicle exhaustion vary among POF patients. The methods of the present invention allow the activation of the remaining follicles in POF patients using the methods of the invention to promote the development of early follicles to the preovulatory stage. This may be followed by the retrieval of mature oocytes for IVF and subsequent pregnancy following embryo transfer.

Due to the delay of child-bearing age in the modern society, many women also are experiencing infertility as the result of diminishing ovarian reserve during aging, e.g. infertile women of from about 40-45 years of age. Although gonadotropin treatments are widely used to promote the development of early antral follicles to the preovulatory stage, many peri-menopausal patients do not respond to the gonadotropin therapy. Because these women still have varying numbers of primordial follicles, they also benefit from the methods of the invention.

Polycystic ovary syndrome is a clinical syndrome characterized by mild obesity, irregular menses or amenorrhea, and signs of androgen excess (eg, hirsutism, acne). In most patients, the ovaries contain multiple cysts. Diagnosis is by pregnancy testing, hormone measurement, and imaging to exclude a virilizing tumor. Treatment is symptomatic. Polycystic ovary syndrome occurs in 5 to 10% of women and involves anovulation or ovulatory dysfunction and androgen excess of unclear etiology. It is usually defined as a clinical syndrome, not by the presence of ovarian cysts. Ovaries may be enlarged with smooth, thickened capsules or may be normal in size. Typically, ovaries contain many 2- to 6-mm follicular cysts and sometimes larger cysts containing atretic cells. Estrogen levels are elevated, increasing risk of endometrial hyperplasia and, eventually, endometrial cancer. Androgen levels are often elevated, increasing risk of metabolic syndrome and causing hirsutism. Over the long term, androgen excess increases risk of cardiovascular disorders, including hypertension.

### Methods of Enhancing Oocyte Maturation

Methods are provided for promoting the development of mammalian ovarian follicles in vitro and in vivo, by contacting follicles with an effective dose of an agent that activates signaling in the mTor pathway, in particular an agent that directly activates mTor, for a period of time sufficient to stimulate the development to antral and preovulatory follicle. Optionally, one or both of an inhibitor of PTEN and an activator of PI3K are also brought into contact with the follicle, at a concentration that is effective to additively induce the follicles to initiate growth.

In some embodiments of the invention, the exposure is performed in vitro, e.g. in an organ or tissue culture, where at least one ovarian follicle is transiently exposed to an effective dose of an agent that activates signaling in the mTor pathway. In some embodiments an intact ovary is thus treated.

The treated follicle may be utilized for in vitro purposes, for example for in vitro fertilization, generation of embryonic stem cells, etc., or may be transplanted to provide for in vivo uses. Transplantation modes of interest include, without limitation, transplantation of one or more follicles, including all or a fraction of an ovary, to a kidney capsule, to Fallopian tubes, to a subcutaneous site, to an ovarian site, e.g. where one ovary has been retained and one has been removed for ex vivo treatment, the one or more treated follicles may be transplanted to the site of the remaining ovary.

In some embodiments, an in vitro method combines treatment with mTor pathway activation with cutting an ovary; and further contacting the ovarian follicles with at least one of a phosphatase and tensin homolog (PTEN) inhibitor, and a phosphatidylinositol 3-kinase (PI3 kinase) activator, which provides for an additive effect to stimulate growth and differentiation of the follicle.

In some embodiments, in vitro treatment is followed by ovarian transplantation, which may be followed by in vitro or in vivo fertilization.

Following exposure to an effective dose of at least one of an agent that disrupts signaling in the MTor pathway, or an agent that acts downstream of disrupted MTor signaling, the individual may be treated with follicular stimulating hormone (FSH) or FSH analogs, including recombinant FSH, naturally occurring FSH in an in vivo host animal, FSH analogs, e.g. FSH-CTP, pegylated FSH, and the like, at a concentration that is effective to initiate follicular growth.

Where the follicles have been stimulated to the antral stage, the individual may be treated luteinizing hormone (LH) or an agonist thereof, which agonists specifically include chorionic gonadotropins, e.g. equine chorionic gonadotropin (eCG), human chorionic gonadotropin (HCG), etc., at an ovulatory dose. In addition, the follicles may be exposed in vivo or in vitro to one or more of c-kit ligand, neurotrophins, vascular endothelial growth factor (VEGF), bone morphogenetic protein (BMP)-4, BMP7, leukemia inhibitory factor, basic FGF, keratinocyte growth factor; and the like.

The dose of an agent that activates signaling in the mTor pathway is sufficient to stimulate pre-antral follicles to induce antral development as described above, and as such, will vary according to the specific agent that is used, the length of time it is provided in the culture, the condition of the follicles, etc. Methods known in the art for empirical determination of concentration may be used. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

As an example, follicle cultures may be contacted with an agent that activates signaling in the mTor pathway at the concentrations previously indicated, for a transient period of time of at least about 1 hour to about 24 hours, and may be from about 6 to about 12 hours. The concentrations may be adjusted to reflect the potency of the agent(s).

Following follicle maturation, the oocytes present in the follicles may be utilized for in vitro purposes. In some embodiments the oocytes are utilized directly, and in others the follicles are contacted with one or more factors to modulate the oocyte maturation, e.g. the cultures may be contacted with a concentration of FSH or FSH analog sufficient to induce oocyte maturation in vitro, where the FSH or FSH analog may be recombinant, modified, native, etc. Following in vitro maturation the oocytes may be fertilized in vitro for implantation; may be fertilized in vitro for generation of stem cell lines; may be utilized without fertilization for various research purposes, and the like.

The follicles may be additionally cultured in the presence of one or more of c-kit ligand (Hutt et al., 2006; Parrott and Skinner, 1999), neurotrophins (Ojeda et al., 2000), vascular endothelial growth factor (Roberts et al., 2007), bone morphogenetic protein (BMP)-4 (Tanwar et al., 2008), BMP7 (Lee et al., 2001), leukemia inhibitory factor (Nilsson et al., 2002), basic FGF (Nilsson et al., 2001), keratinocyte growth factor (Kezele et al., 2005), and the like, where the factor(s) may be added in conjunction with an agent that activates signaling in the mTor pathway. For example, an LH agonist, including eCG and/or HCG may be administered following oocyte maturation by FSH.

In other embodiments the follicles may be transplanted to an animal recipient for maturation. As described above, methods are known in the art for transplantation of ovaries or fragments thereof at an ovarian site, at a kidney site, at a sub-cutaneous site, etc. are known in the art and may find use. Where the ovarian tissue is transplanted to an ovary, fertilization may proceed without additional in vitro manipulation. Where the ovarian tissue is transplanted to a non-ovarian site, e.g. a sub-cutaneous site, the oocytes may be subsequently removed for in vitro fertilization. The recipient may provide endogenous FSH for maturation of the oocytes, or may be provided with exogenous FSH or FSH analog for that purpose, including recombinant, long-acting FSH-CTP, and the like.

In other embodiments, the exposure is performed in vivo, locally to the ovary or systemically administered to an individual. The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized. The individual is typically contacted with an effective concentration for at least about 6 hours, usually at least about 12 hours, and may be for at least about 1 day and not more than about one week, usually not more than about 3 days.

The compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The effective dose of an agent that activates signaling in the mTor pathway can be administered in a variety of different ways. Examples include administering a composition via oral, topical, intraperitoneal, intravenous, intramuscular, subcutaneous, subdermal, transdermal, intra-ovarian methods. In pharmaceutical dosage forms, the compounds may be administered in the form of their pharmaceutically acceptable salts, or they may also be used alone or in appropriate association, as well as in combination with other pharmaceutically active compounds.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

Following such exposure, the individual may be treated with recombinant FSH or FSH analogs, including, without limitation, naturally occurring FSH in an in vivo host animal, FSH analogs such as FSH-CTP, single chain analogs, pegylated FSH, and the like, at a concentration that is effective to release a mature oocyte. The individual may also be treated with an LH agonist as described above. Alternatively, the oocytes may be removed from the ovary and utilized for in vitro manipulation as described above.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Promotion of Ovarian Follicle Growth Following mTOR Activation: Synergistic Effects of AKT Stimulators

Mammalian ovaries consist of follicles as basic functional units. During initial recruitment of follicles, unknown intraovarian mechanisms stimulate or release a small number of dormant primordial follicles to initiate growth. Once entering the growing pool, ovarian follicles mature through primary, secondary, and antral stages to become preovulatory follicles containing mature oocytes. Mammalian target of rapamycin (mTOR) is a serine/threonine kinase conserved from flies to mammals and part of the multi-protein mTORC1 complexes. Under the influence of nutritional factors, stress, oxygen, energy and other clues, the rapamycin-sensitive mTORC1 complex positively regulates cell growth and proliferation by promoting diverse anabolic processes, including biosynthesis of proteins, lipids and organelles, and by limiting catabolic processes such as autophagy. In contrast, the tumor suppressor tuberous sclerosis complex 1 (TSC1) or 2 (TSC2) negatively regulates mTORC1 activity. Inactivating mutations of TSC1 or TSC2 result in tuberous sclerosis complex (TSC), a disease characterized by numerous benign tumors containing enlarged cells.

Studies using mutant mice indicated that oocyte-specific deletion of TSC1 or TSC2 promotes the growth of all primordial follicles in neonatal animals, leading to the exhaustion of the entire follicle pool, followed by a premature ovarian failure phenotype. Likewise, oocyte-specific deletion of the PTEN gene, upstream of AKT signaling, also increases AKT activity, followed by global activation of dormant ovarian follicles. Of interest, double deletion of TSC1 and PTEN leads to synergistic enhancement of oocyte growth and follicle activation when compared with singly mutated mice. For larger follicles, mutant mice with disrupted TSC1 in granulosa cells of secondary follicles also exhibit enhanced follicle growth, leading to increased ovulatory capacity and delivery of more pups, followed by a premature ovarian failure phenotype.

Taking advantage of the availability of an mTOR activator MHY1485, we stimulated secondary follicle growth in juvenile mice using an in vitro activation-grafting approach and derived preovulatory follicles containing mature oocytes.

### Results:

MHY1485 Treatment Stimulated Phosphorylation of mTOR Pathway Proteins.

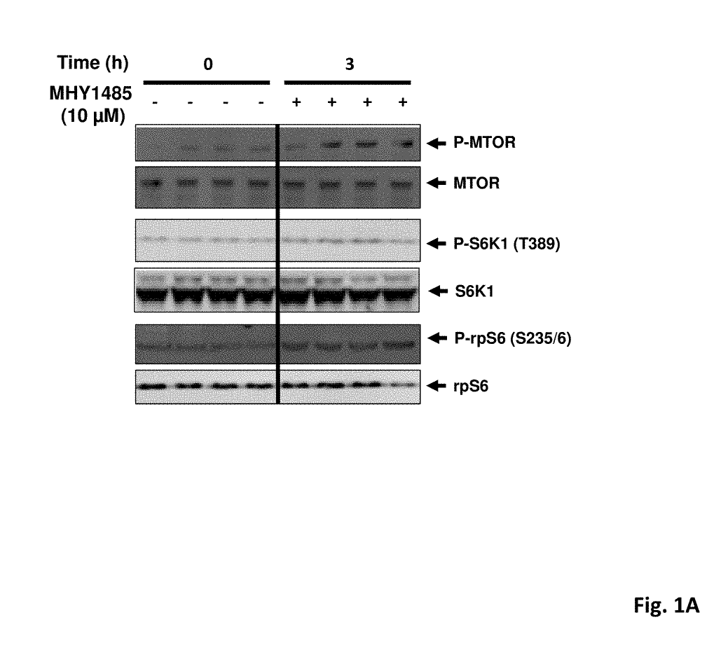

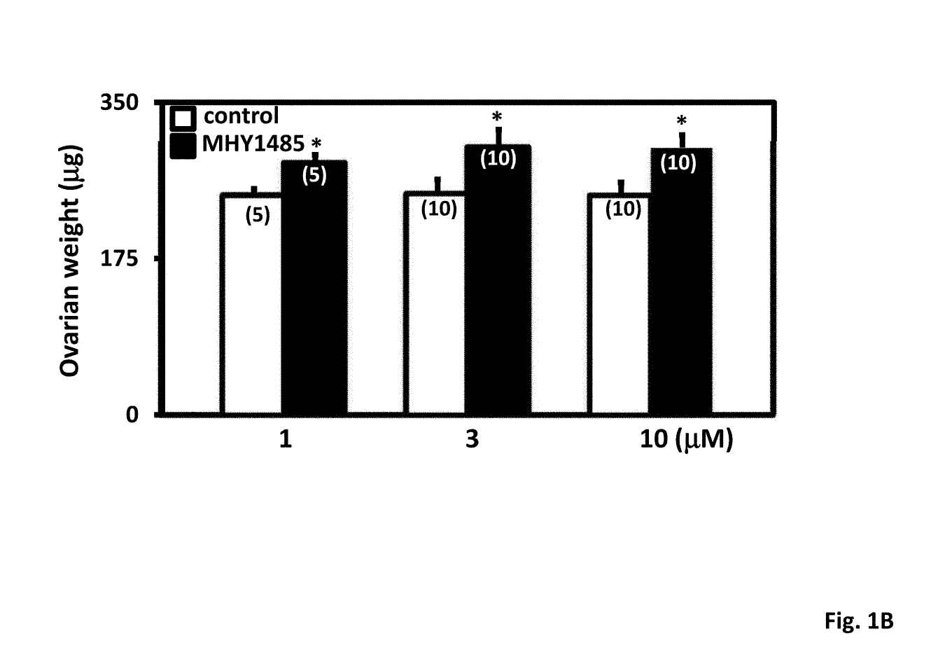

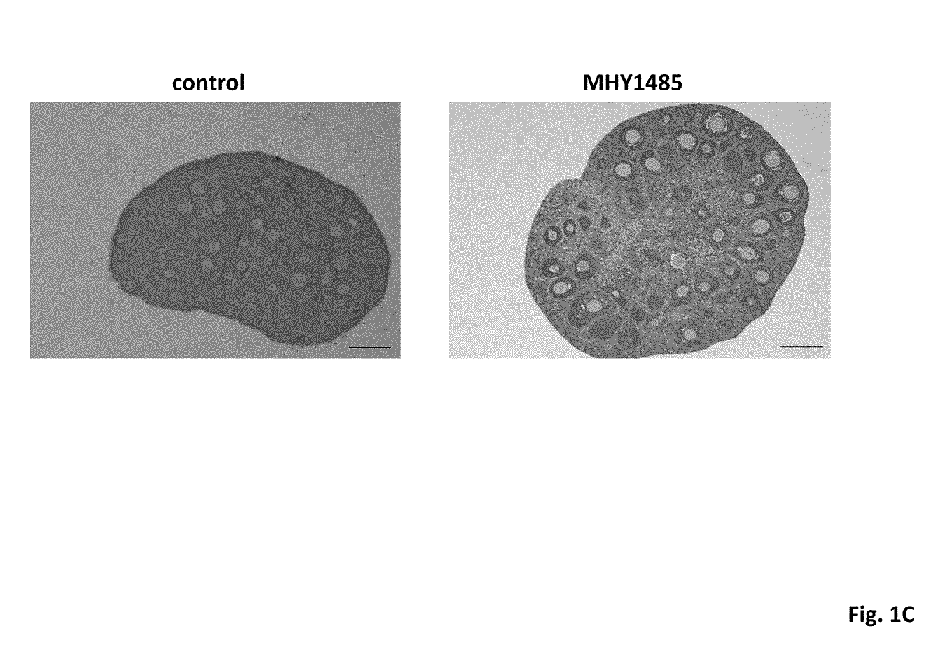

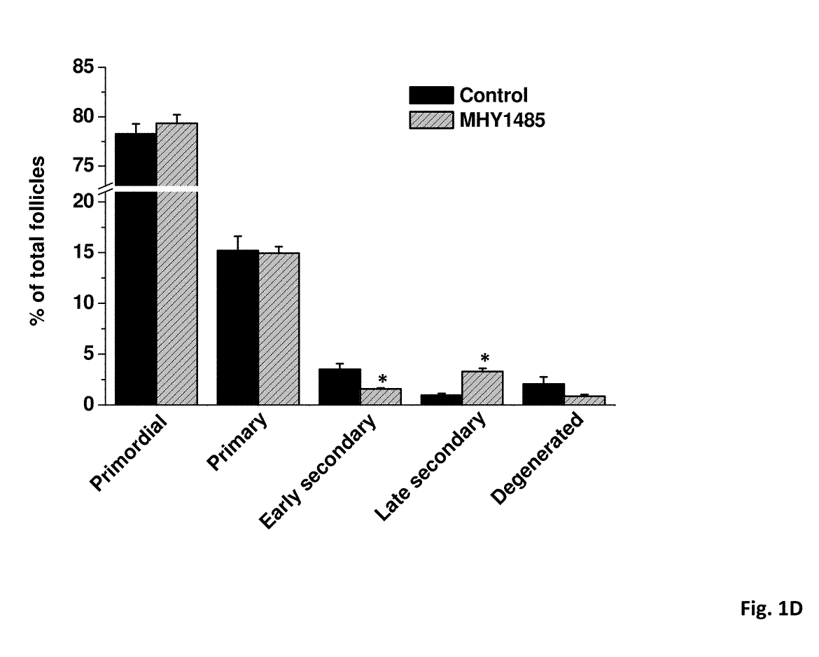

Based on recent studies showing the ability of MHY1485 to activate the mTOR pathway in rat hepatocyte and PC3 cell line, we investigated ovarian phosphorylation of mTOR and downstream proteins in this signaling pathway after MHY1485 treatment. Ovaries from day 10 mice were incubated for 3 h with 10 μM of MHY1485 before immunoblotting analyses. As shown in FIG. 1A, treatment with MHY1485 increased phospho-mTOR levels without affecting total mTOR content. Activated mTORC1 complex phosphorylates Thr389 in ribosomal S6 kinase (S6K), thereby activating it to subsequently phosphorylate ribosomal protein S6 (rpS6) and promote ribosome biogenesis. We further monitored S6K1 and rpS6 phosphorylation in ovarian tissues. As shown in FIG. 1A, MHY1485 treatment also increased the phosphorylation of downstream S6K1 and rpS6 proteins without affecting total S6K1 and rpS6 levels. These findings demonstrate the ability of MHY1485 to stimulate the mTOR signaling pathway in the ovary.

Treatment with MHY1485 Promoted Follicle Growth In Vitro and In Vivo:

We treated ovaries from day 10 mice with increasing doses of MHY1485 using an explant culture model. As shown in FIG. 1B, treatment of ovaries for 4 days led to dose-dependent increases in ovarian weights. Histological analyses (FIG. 1C) and counting of follicles (FIG. 1D) indicated enhancement of follicle growth from early secondary to the late secondary stage.

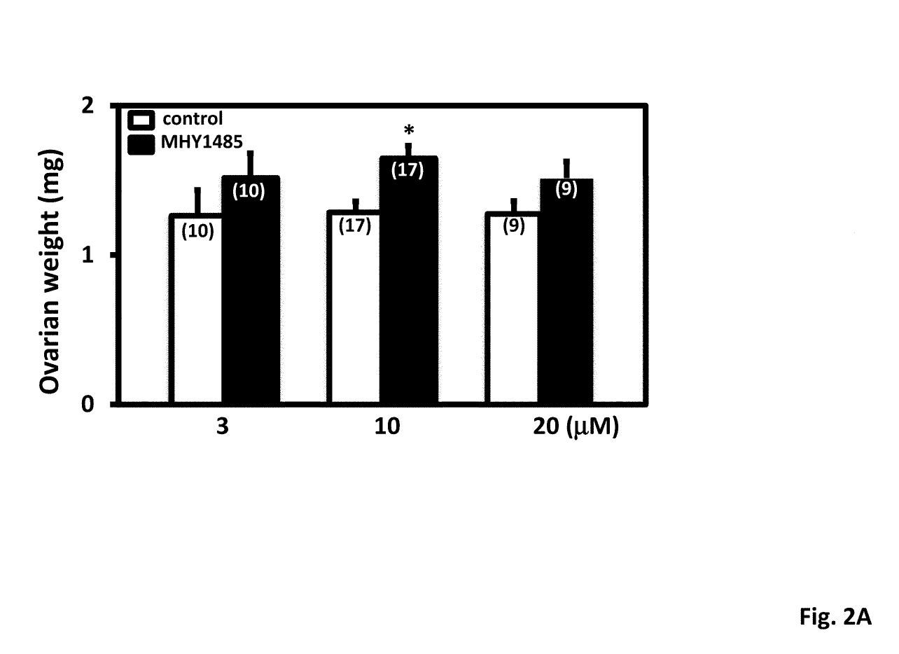

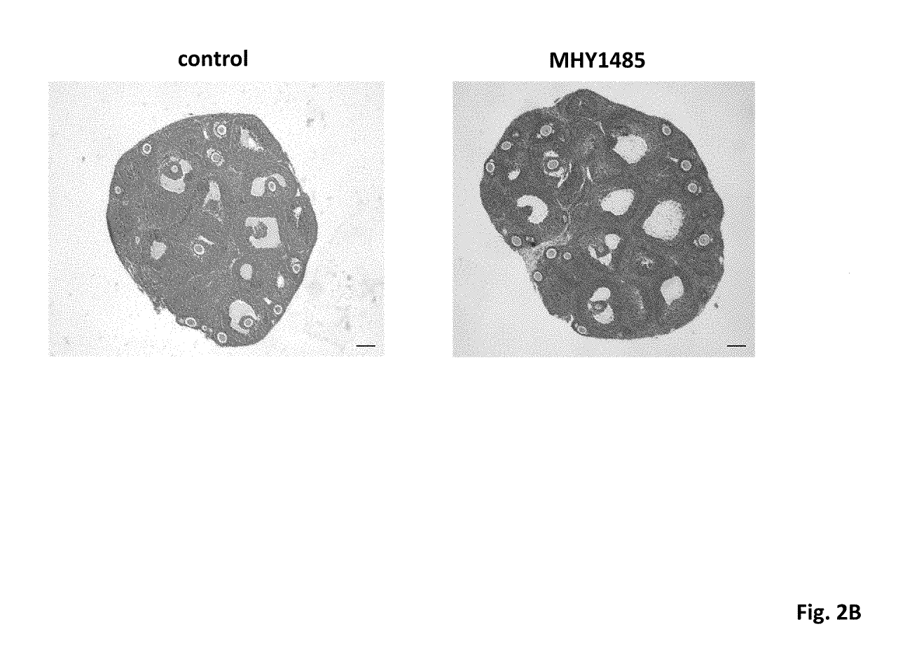

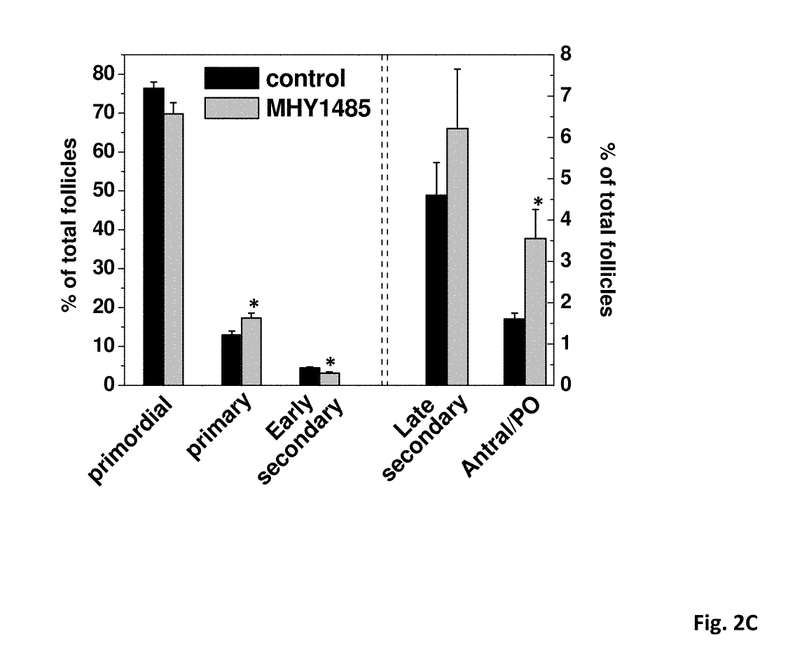

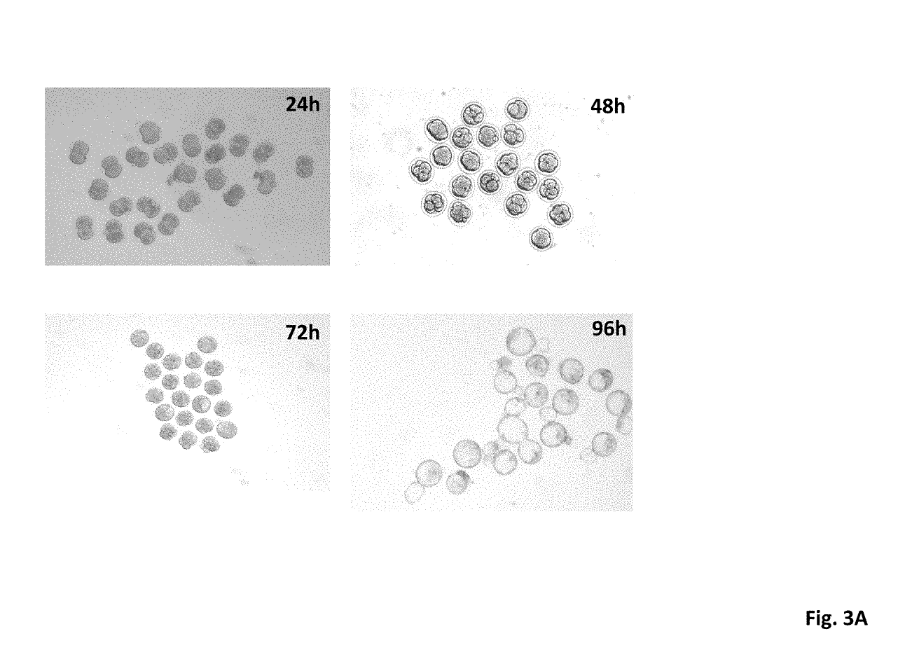

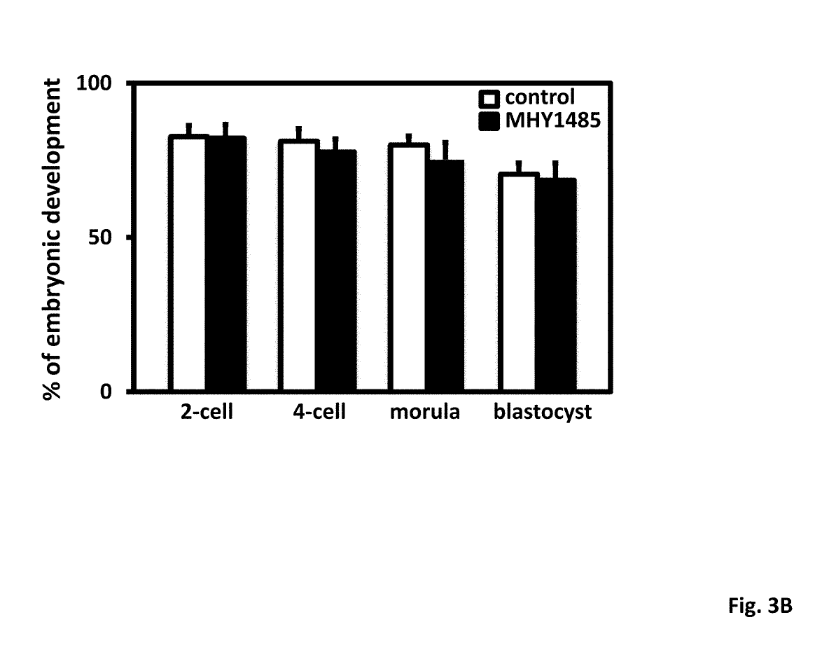

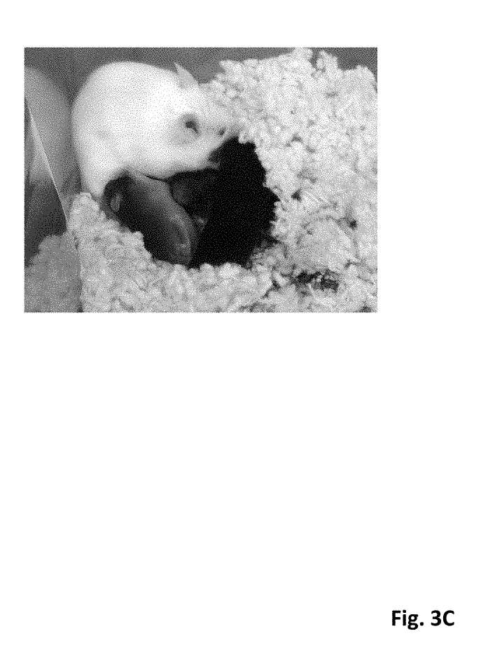

We further treated day 10 ovaries with MHY1485 for 2 days in vitro, followed by grafting them into adult hosts treated daily with FSH for 5 days. As shown in FIG. 2A, treatment with MHY1485 increased graft weights. Following histological analyses (FIG. 2B) and follicle counting (FIG. 2C), increases in the development of antral/preovulatory follicles were apparent, together with a decrease of early secondary follicles and an increase of primary follicles. Using this model, we further treated the hosts with eCG for 2 days to promote the growth of preovulatory follicles, followed by an injection of hCG to promote oocyte maturation. At 12 h after hCG injection, mature oocytes were punctured from preovulatory follicles for in vitro fertilization. As shown in FIG. 3A, oocytes obtained from MHY1485-pretreated ovaries could develop into blastocysts. As compared with mature oocytes obtained from day 25 mice without MHY1485 treatment (controls), comparable early embryonic development was apparent based on the percentage of oocytes developing into each embryonic stage (FIG. 3B). Some of the 2-cell embryos derived from MHY1485-pretreated grafts were transferred into pseudopregnant surrogate mothers and healthy pups were delivered (FIG. 3C).

Treatment with the mTOR Activator Augmented Follicle Growth Promoted by AKT Stimulators:

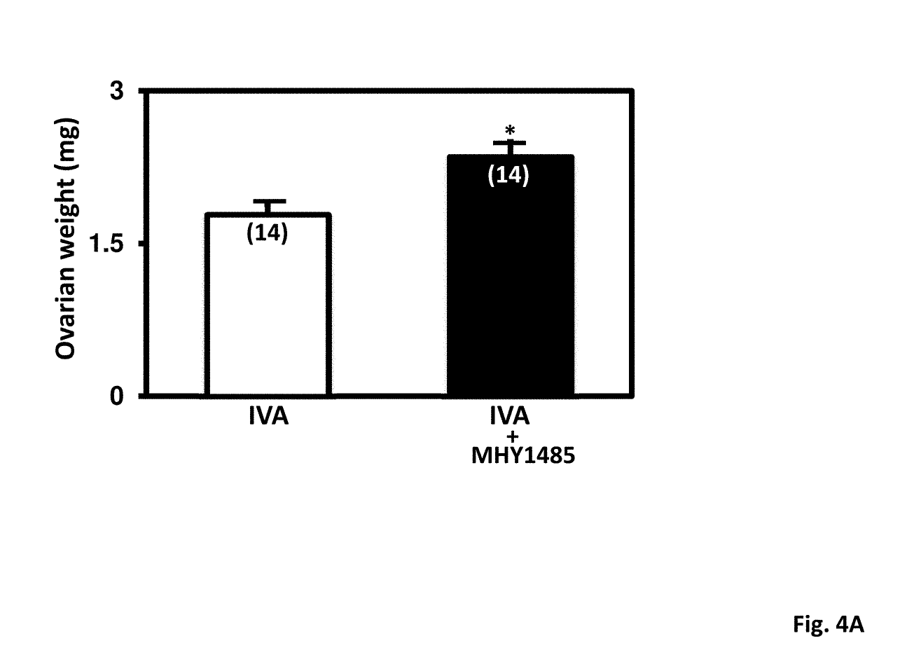

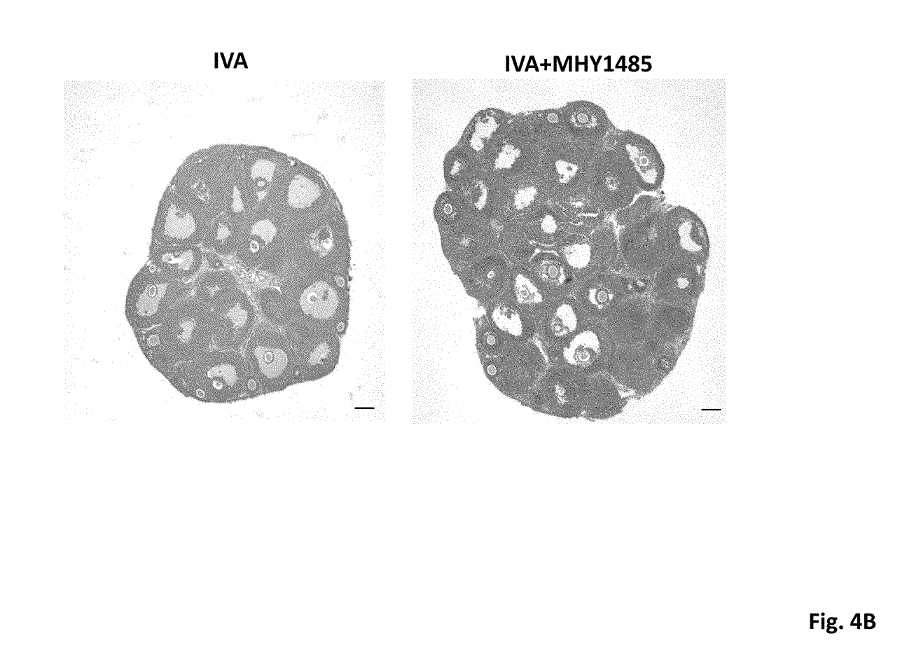

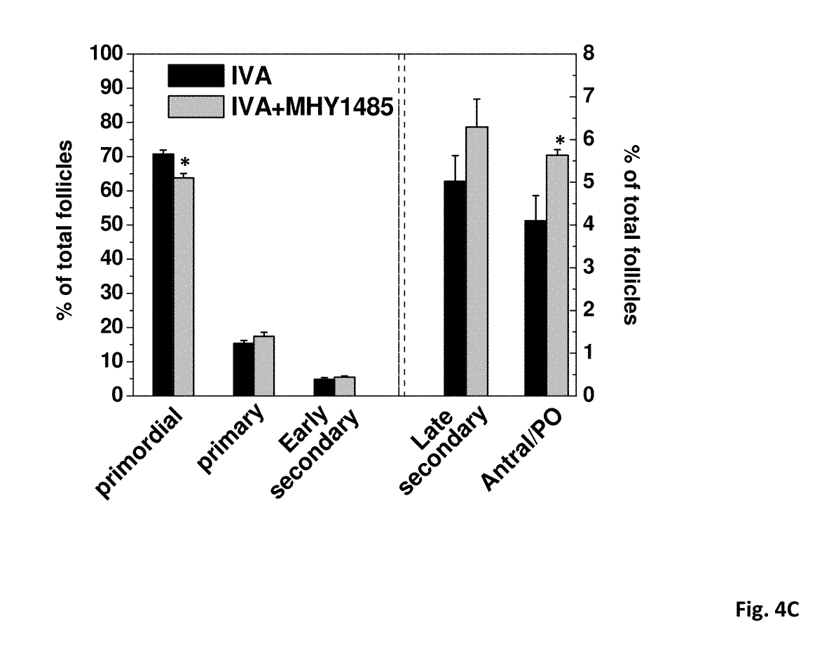

Our earlier findings indicated the ability of AKT stimulators including PTEN inhibitors and phosphoinositol-3-kinase activators to promote secondary follicle growth. We, therefore, tested the combined effects of treating day 10 ovaries with both mTOR activator and AKT stimulators. Ovaries from day 10 mice were treated with optimal doses of the PTEN inhibitor bpv(hopic) and 740YP (an activator for phosphoinositol-3-kinase) routinely used in our in vitro activation (IVA) protocol with or without MHY1485. As shown in FIG. 4A, co-treatment with MHY1485 and the IVA drugs further augmented graft weights. Histological analyses (FIG. 4B) and follicle counting (FIG. 4C) indicated increases in antral/preovulatory follicles, accompanied by a decrease of primordial follicles.

Our studies demonstrated the ability of an mTOR activator to stimulate the phosphorylation of mTOR and downstream proteins, to enhance secondary follicle growth in ovarian explant cultures, and to promote the generation of antral/preovulatory follicles in allografts. In addition to the PTEN-AKT-FOXO3 signaling pathway, suppression of mTORC1 activity by the TSC1-TSC2 complex in oocytes has been shown to be a prerequisite for maintaining the dormancy of primordial follicles based on extensive studies using mice with oocyte-specific deletion of TSC1 and TSC2 genes. Both PTEN and TSC1/2 suppress phosphorylation/activation of rpS6, but by regulating the phosphorylation of distinct threonine residues in S6K1. These earlier findings demonstrate a role for AKT and mTOR1 signaling pathways in the regulation of primordial follicle dormancy (Adhikari & Liu (2010) Cell Cycle 9, 1673-1674).

For secondary follicles, disruption of Tsc1 (Huang, L. et al. (2013) PLoS One 8, e54052), or activation of the AKT (Fan et al. (2008) Mol Endocrinol 22, 2128-2140) signaling pathway in granulosa cells of secondary follicles also promotes follicle development. Augmentation of follicle growth following treatment with mTOR and AKT signaling activators described herein likely reflect the stimulation of follicle growth mediated by granulosa cells due to the short duration of in vivo grafting.

Analyses of follicle dynamics herein demonstrated that short-term exposure to the mTOR activator promotes the growth of early secondary follicles to the antral/preovulatory stage in grafts. After stimulation of secondary follicles with MHY1485 to derive antral follicles, further treatment of animals with gonadotropins allowed the generation of multiple preovulatory follicles containing mature oocytes capable of developing into blastocysts and viable pups. The present findings show that short-term exposure to mTOR signaling activators, similar to AKT signaling stimulators, provides a basis for infertility therapies. In contrast to the ability of the mTOR activator to promote follicle growth described here, long-term injections with rapamycin (an inhibitor of mTOR signaling) lead to the suppression of follicle development in PTEN mutant mice (Adhikari et al. (2013) PLoS One 8, e53810) and prolong the fertile lifespan of aging rats by arresting follicle growth (Zhang, et al. (2013) Gene 523, 82-87).

Although fertility is compromised in patients with primary ovarian insufficiency and middle-aged sub-fertile women, their ovaries still contain small number of preantral follicles. Our earlier studies demonstrated that short-term exposure of human ovarian fragments with AKT stimulators (PTEN inhibitors and PI3K activators) promotes follicle growth and allow the generation of mature oocytes in ovarian grafts in a subpopulation of patients with primary ovarian insufficiency, leading to a new infertility treatment approach. The present data further demonstrated the augmentation of follicle growth in ovarian grafts pre-incubated with both AKT stimulators and an mTOR activator. This transient and ovary-specific exposure to mTOR activators in vitro, when combined with treatment with AKT stimulators, improves the success of infertility treatment as compared with the use of AKT stimulators alone.

### Methods:

Animals:

CD-1 and B6D2F1 mice were purchased from Charles River Laboratories (Wilmington, Mass.) and housed in animal facility of Stanford University under 12 h light/dark with free access to water and food. Mice were treated in accordance with guidelines of local Animal Research Committee.

Immunoblotting Analysis:

Ovaries from mice at day 10 of age were treated with MHY1485 (Millipore, Bedford, Mass.) for 3 h and proteins were extracted using M-PER Mammalian Protein Extraction Reagent (Thermo, Rockford, Ill.) containing a protease inhibitor cocktail (Thermo). Protein concentrations in supernatants were determined by the bicinchoninic acid method (Pierce, Rockford, Ill., USA). Equal amounts of protein lysates were loaded on 4-12% NuPAGE Bis-Tris gels (Invitrogen, Carlsbad, Calif.) in MOPS buffer and transferred to 0.45 μM pore nitrocellulose membranes (LI-COR, Lincoln, Nebr., USA). First antibodies were from Cell Signaling (Beverly, Mass.) and rabbit secondary antibodies from LI-COR. Images were generated using a LI-COR Odyssey infrared imager.

Ovarian Explant Culture and Follicle Counting:

Ovaries from day 10 mice were placed on culture plate inserts (Millipore) and cultured in 400 μl of DMEM/F12 containing 0.1% BSA, 0.1% Albumax II, insulin-transferrin-selenium, 0.05 mg/ml L-ascorbic acid and penicillin-streptomycin under a membrane insert to cover ovaries with a thin layer of medium. Ovaries were treated with 1-10 μM of MHY1485 and cultured for 4 days with medium change after 2 days of culture. At the end of culture, ovaries were fixed with formalin before weighing. Some ovaries were paraffin-embedded and cut into continuous sections. Sections were stained with hematoxylin and eosin for follicle counting, and only follicles with clearly stained oocyte nucleus were counted to avoid recounting of the same follicle.

Ovarian Tissue Grafting:

Paired ovaries from day 10 mice were cultured on plate culture inserts in MEMα medium containing 3 mg/ml BSA, 0.23 mM sodium pyruvate, 50 μg/ml vitamin C, 30 mIU/ml FSH, 50 mg/L streptomycin sulfate and 75 mg/L penicillin G. Ovaries were treated with 3-20 μM MHY1485 for 48 h with medium changes after 24 h of culture. Paired ovaries (without or with MHY1485 treatment) from the same donor were grafted under kidney capsules of the same adult ovariectomized hosts (9-10 weeks of age) for 5 days with daily FSH injections (1 IU/animal). At the end of transplantation, grafts were collected for weight determination and histological analysis. For some animals, ovaries from day 10 mice were treated with IVA drugs (PTEN inhibitor: (bpv(hopic) at 30 μM for the first day and an activator for phosphoinositol-3-kinase 740YP at 150 μg/mL for two days) before grafting.

In Vitro Fertilization and Embryo Transfer:

Ovaries from B6D2F1 mice at 10 days of age were treated with 10 μM MHY1485 for 2 days, followed by transplantation into kidney capsules of hosts for 5 days. At day 5 after transplantation, animals were treated with 10 IU equine chorionic gonadotropin (eCG) for 48 h, followed by an injection of 10 IU human chorionic gonadotropin (hCG). Twelve hour later, grafts were collected and oocytes were retrieved in the M2 medium (Cytospring, Mountain View, Calif.). As controls, B6D2F1 mice at day 25 of age were treated with 5 IU eCG for 48 h, followed by 5 IU hCG before oocyte retrieval. For in vitro fertilization, sperm from B6D2F1 male mice were collected into human tubal fluid medium (Cytospring) and pre-incubated for 1 h at 37 C. Oocytes were then fertilized with sperm (2-3×10⁵/ml) for 6 h, and inseminated oocytes were transferred into KSOM medium (Cytospring) for development into blastocysts. For embryo transfer, two-cell embryos were transferred into oviducts of pseudopregnant, 8-week-old CD1 nice pre-mated with vasectomized males of the same strain.

All publications and patent applications cited in this specification are herein incorporated by reference as if each individual publication or patent application were specifically and individually indicated to be incorporated by reference. The publications discussed herein are provided solely for their disclosure prior to the filing date of the present application. Nothing herein is to be construed as an admission that the invention is not entitled to antedate such a disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of promoting the development of mature oocytes, the method comprising:
contacting ovarian tissue in vivo or in vitro with an effective dose of an agent that activates signaling in the mechanistic target of rapamycin (mTor) pathway for a period of time sufficient to promote the development of a mature oocyte.

2. The method of claim 1, wherein the mature oocyte is contained within a pre-ovulatory follicle.

3. The method of claim 2, wherein the pre-ovulatory follicle is a secondary follicle.

4. The method of claim 2 wherein the pre-ovulatory follicle is an antral follicle.

5. A method of increasing phosphorylation of ribosomal S6 kinase 1 (S6K1) and ribosomal protein S6 (rpS6) in ovarian tissue, the method comprising:
contacting ovarian tissue in vivo or in vitro with an effective dose of an agent that activates signaling in the mechanistic target of rapamycin (mTor) pathway for a period of time sufficient to increase phosphorylation of ribosomal S6 kinase 1 (S6K1) and ribosomal protein S6 (rpS6).

6. The method of claim 1, wherein the ovarian tissue is in an intact ovary.

7. The method of claim 1, wherein the contacting is performed in vivo.

8. The method of claim 1, wherein the contacting is performed in vitro.

9. The method of claim 1, wherein the ovarian tissue is in in vitro tissue culture.

10. The method of claim 1, wherein the ovarian tissue is human.

11. The method of claim 1, wherein the ovarian tissue is from a mammal selected from the group consisting of mice, canines, felines, rabbits, pigs, cows, buffalos, sheep, horses, pandas, chimpanzees and gorillas.

12. The method of claim 1, wherein the agent directly activates mTor.

13. The method of claim 1, wherein the agent is selected from the group consisting of MHY1485, 3BDO, and CL316,243.

14. The method of claim 1, wherein the effective dose is from 0.1 μM to about 1 mM.

15. The method of claim 1, wherein the ovarian tissue is contacted for one hour to four days.

16-36. (canceled)
